SEQUENCE LISTING (25 sequences)

SEQ ID NO: 1 - DNA, 498 bp, Streptococcus thermophilus
atggaacaagaagtttttgttaaggcatatgaaaaggtaaggccaattgtacttaaggcttttaggcaatactttattcagctttgggatcaagctgacatggagcaagaggcgatgatgactttgtatcagcttttaaaaaagtttcctgatttagagaaagatgatgataagttacgtcgttactttaaaactaagtttaggaatcgacttaatgatgaagtgaggcggcaggagtcagtaaaacgtcaagctaatagacagtgctatgttgaaatttcagatattgccttttgtattcccaataaggagctagatatggttgatagacttgcttatgatgaacagcttaatgcatttcgtgagcagttatcatcggaagattctcttaagttggatcgattgttgggtggtgaatgctttaggggaaggaaaaagatgatacgagagttaagattttggatggttgacttcgatccatgtaatgaagaagactga

SEQ ID NO: 2 - protein, 165 aa, Streptococcus thermophilus
MEQEVFVKAYEKVRPIVLKAFRQYFIQLWDQADMEQEAMMTLYQLLKKFPDLEKDDDKLRRYFKTKFRNRLNDEVRRQESVKRQANRQCYVEISDIAFCIPNKELDMVDRLAYDEQLNAFREQLSSEDSLKLDRLLGGECFRGRKKMIRELRFWMVDFDPCNEED

SEQ ID NO: 3 - DNA, 258 bp, Streptococcus thermophilus
cttcaaggtctagtcctctcttttatgacgaatactgtttattgaaaaattgtaacataaagaaaacggtttttcatttttttatgagtataaaatgagatttttttctgaattttagaaataatatacattaggaattaccattcgggacatatagccactttttgggacgctagctctgatagagacaattgaatgctatactaaagatgtgattgagagatcacacgataaaaattttaggaggtagttgccatg

SEQ ID NO: 4 - protein, 30 aa, Streptococcus thermophilus
SGWMDYINGFLKGFGGQRTLPTKDYNIPQV

SEQ ID NO: 5 - protein, 53 aa, Streptococcus thermophilus
MANNTINNFETLDNHALEQVVGGSGWMDYINGFLKGFGGQRTLPTKDYNIPQV

SEQ ID NO: 6 - DNA, 38 bp, Artficial Sequence
  CBF primer
agtgtaactgcagaatacttgcaggtctatcgatcgat

SEQ ID NO: 7 - DNA, 39 bp, Artficial Sequence
  CBL primer
tttggcggatctcataaggacctcctcataaacctattc

SEQ ID NO: 8 - DNA, 39 bp, Artficial Sequence
  CXPF primer
cgctttctgcagctatcactctaatacaatcctgtggaa

SEQ ID NO: 9 - DNA, 44 bp, Artficial Sequence
  CXPL primer
ttggcggatctcattgaacctccaataataaatataaattctgt

SEQ ID NO: 10 - DNA, 33 bp, Artficial Sequence
  BPF primer
gtaaatctgcagcttcaaggtctagtcctctct

SEQ ID NO: 11 - DNA, 40 bp, Artficial Sequence
  BPL primer
ttggcggatctcatggcaactacctcctaaaatttttatc

SEQ ID NO: 12 - DNA, 35 bp, Artficial Sequence
  LCB primer
tatgaggaggtccttatgagatccgccaaaaacat

SEQ ID NO: 13 - DNA, 36 bp, Artficial Sequence
  LR primer
catatggctcgagtgcactctcagtacaatctgctc

SEQ ID NO: 14 - DNA, 43 bp, Artficial Sequence
  LXP primer
tattggaggttcaatgagatccgccaaaaacataaagaaaggc

SEQ ID NO: 15 - DNA, 45 bp, Artficial Sequence
  LBP primer
taggaggtagttgccatgagatccgccaaaaacataaagaaaggc

SEQ ID NO: 16 - DNA, 27 bp, Artficial Sequence
  P1 primer
gtttgagttgccatggcaactacctcc

SEQ ID NO: 17 - DNA, 39 bp, Artficial Sequence
  P2 primer
attaggatccttcaaggtctagtcctctcttttatgacg

SEQ ID NO: 18 - DNA, 38 bp, Artficial Sequence
  X1 primer
attatctagaccaagaattactggaaacacaatagagg

SEQ ID NO: 19 - DNA, 35 bp, Artficial Sequence
  X2 primer
ggaggttccatggaacaagaagtttttgttaaggc

SEQ ID NO: 20 - DNA, 27 bp, Artficial Sequence
  EC1 primer
gaggcatcattggaagaatagagcagc

SEQ ID NO: 21 - DNA, 54 bp, Artficial Sequence
  EC2 primer
aagcttaagatctagagctcgaggatcaaaaactagagagaagattgccgtcag

SEQ ID NO: 22 - DNA, 49 bp, Artficial Sequence
  EC3 primer
agcatgcatatgcatccggagtcctagcttgtttcagtttgtctcaatg

SEQ ID NO: 23 - DNA, 24 bp, Artficial Sequence
  EC4 primer
ccatcccttaaaccgaatggcacc

SEQ ID NO: 24 - DNA, 26 bp, Artficial Sequence
  Kana-F primer
atcctcgagctctagatcttaagctt

SEQ ID NO: 25 - DNA, 22 bp, Artficial Sequence
  Kana-R primer
actccggatgcatatgcatgct

(19) United States
(12) Patent Application Publication
(10) Pub. No.: US 2010/0047384 A1
(43) Pub. Date: Feb. 25, 2010

(54) METHODS OF PRODUCING TRANSFORMATION COMPETENT BACTERIA

(75) Inventors: Leiv Sigve Havarstein, AS (NO); Hilde Steinmoen, Fyllingsdalen (NO); Trinelise Blomqvist, Svinndal (NO)

Correspondence Address: KNOBBE MARTENS OLSON & BEAR LLP, 2040 MAIN STREET, FOURTEENTH FLOOR, IRVINE, CA, 92614, US

(73) Assignee: NORWEGIAN UNIVERSITY OF LIFE SCIENCES, As (NO)

(21) Appl. No.: 12/299,587

(22) PCT Filed: May 8, 2007
(86) PCT No.: PCT/GB2007/001664

§ 371 Date: Oct. 23, 2009

(30) Foreign Application Priority Data
May 5, 2006 (GB) 0608966.8

(51) Int. Cl. A23L 1/28; C12N 15/74; C12N 15/63; C12N 1/21

(52) U.S. Cl. 426/7; 435/476; 435/320.1; 435/252.3; 426/61; 435/29

(57) ABSTRACT

A method of producing transformation competent bacteria, comprising the steps of: (i) transforming a bacteria that is not naturally transformation competent with a plasmid, wherein said plasmid comprises a comX gene sequence encoding a ComX protein or functional part or derivative or variant thereof under the regulatory control of a promoter which is inducible by a transcription initiator, (ii) contacting said transformed bacteria with said transcription initiator to initiate transcription of said comX gene sequence is provided. Also provided are plasmids, transformed bacteria, transformation competent bacteria, mutant bacteria and food products comprising said mutant bacteria.

The present invention relates to methods of generating transformation competent bacteria, particularly streptococci, methods for producing mutant bacteria from such transformation competent bacteria, the bacteria thus produced, vectors or plasmids for producing such transformation competent bacteria and the use of the mutant bacteria in the generation of food products.

Bacteria that are competent for natural genetic transformation are able to take up naked DNA from the environment and incorporate it into their genomes by homologous recombination. Several streptococcal species belonging to the mitis, anginosus and mutans phylogenetic groups have been shown to possess this property (Håavarstein et al., 1997, J. Bacteriol., 179, p 6589-6594; Clayerys & Håvarstein, 2002, Front. Biosci., 7, d1798-1814), but the phenomenon has never been demonstrated in most members of the genus.

One of the best studied naturally competent bacteria is Streptococcus pneumoniae. In this species, and other streptococci shown to be naturally transformable, competence is not a constant property, but a transient state regulated by a quorum-sensing mechanism consisting of ComABCDE (Claverys & Håvarstein, 2002, supra). ComC encodes the precursor of a secreted peptide pheromone, the competence stimulating peptide (CSP), which triggers development of the competent state when its external concentration in a pneumococcal culture reaches a critical threshold (Håvarstein et al., 1995, Proc. Natl. Acad. Sci. USA, 92, p 11140-11144).

CSP is secreted by ComAB and acts through a two-component signal transduction pathway consisting of the histidine kinase ComD and the cognate response regulator ComE (Claverys & Håvarstein, 2002, supra; Håvarstein et al., 1996, Mol. Microbiol., 21, p 863-869; Pestova et al., 1996, Mol. Microbiol., 21, p 853-862). When the external concentration of CSP in a pneumococcal culture reaches about 10 ng/ml, early and late competence genes are expressed, resulting in development of the competent state. The early genes are regulated by ComE, which initiates transcription from promoters containing a conserved direct-repeat motif (P_{E}), whereas the alternative sigma factor ComX is needed for expression of the late genes (Lee & Morrison, 1999, J. Bacteriol., 181, p 5004-5016; Peterson et al., 2004, Mol. Microbiol., 51, p 1051-1070; Dagkessamanskaia et al., 2004, Mol. Microbiol., 51, p 1071-1086). Late genes share an 8-bp sequence in their promoter regions that is specifically recognized by a ComX-directed RNA-polymerase holoenzyme (Lee & Morrison, 1999, supra). Circumstantial evidence indicates that ComX belongs to the early genes and therefore depends on ComE for its expression (Claverys & Håvarstein, 2002, supra).

The fourteen pneumococcal proteins known to be necessary for uptake of extracellular DNA, and subsequent incorporation of this DNA into the recipient's genome, are all encoded by late genes (Peterson et al., 2004, supra; Dagkessamanskaia et al., 2004, supra). Recent genome sequencing has shown that the ComX regulon appears to be present in all streptococcal species. However, in for example S. thermophilus, whilst the late genes involved in binding, uptake and recombination of DNA are present on the genome and ComX genes are present, this bacterium is not naturally transformation competent. The early genes that control competence development in the pneumococcus and several related streptococci are missing in S. thermophilus and thus it is not known whether the bacterium may be made transformation competent or how this might be achieved. Thus, the late genes of streptococcal species not known to be competent may serve other functions, or represent non-functional relicts inherited from a competent ancestor.

Transformation competent bacteria are extremely desirable for the production of mutant bacteria which have altered properties relative to their parent strain. For example various streptococci are exceedingly important in the food industry. The food industry is continuously working to improve the properties of the strains that are used, but when the strains are not naturally transformation competent they have been hampered in this development.

Streptococcus thermophilus is, for example, of major importance in the food industry and is widely used for the manufacture of dairy products (yoghurt and Swiss or Italian-type cheeses) with an annual market value of approximately $40 billion making S. thermophilus a species of major economic importance. The dairy industry is continuously working to improve the properties of S. thermophilus starter strains. Even though the fermentation properties of this bacterium have been gradually improved by classical methods, there is great potential for further improvement through genetic engineering. However, until now, suitable genetic tools have not been available. Traditionally, mutants with sought after properties have been made by subjecting a culture of the parental strain to UV radiation or mutagenic chemicals followed by identification of the mutant carrying the desired phenotype. An effective protocol for “finding the needle in a haystack”, i.e. the bacterial cell harbouring the correct mutation, is usually not available. It is therefore extremely labour intensive and often impossible to make mutant strains with novel properties by this classical route. Besides, treatment of the S. thermophilus cells with UV radiation or mutagenic chemicals introduces mutations randomly all over the bacterial genome, potentially giving rise to a number of unwanted physiological changes in the parental strain.

Targeted mutations, such as for example, construction of knock-out mutants, can be made in S. thermophilus by applying standard genetic methods, but the tools are poorly developed and inefficient compared to other lactic acid bacteria. In addition, new traits can in principle be introduced on recombinant plasmids transformed into S. thermophilus cells by electroporation. The major drawback with these methods is that they cannot be regarded as food grade. Finding the correct mutant among 10⁹ wild type cells requires the presence of a marker gene, in most cases an antibiotic resistance gene, which renders the resulting mutant strain unsuitable for human consumption.

Surprisingly it has now been found that bacterial strains such as S. thermophilus may be modified to make them transformation competent which makes genetic manipulations easy, rapid and highly efficient. This makes it possible to construct food grade mutants of bacteria, particularly S. thermophilus, which opens up exciting new possibilities that will benefit consumers as well as the dairy industry.

As described hereinafter, we have developed a new highly efficient method for genetic manipulation of bacteria such as S. thermophilus based on the natural properties of the bacteria.

In one embodiment of this method, as described in the Examples, a system for overexpression of the alternative sigma factor ComX was constructed in which the comX gene was cloned behind a bacteriocin promoter on a vector termed pTRKH2. A peptide pheromone regulates bacteriocin production in S. thermophilus by a quorum-sensing system similar to the one that controls the development of natural transformation in S. pneumonia. By adding the peptide pheromone to a culture of S. thermophilus growing under the right conditions and harbouring the recombinant helper plasmid described above, competence for natural transformation was induced due to high expression of ComX. By adding a DNA fragment containing the desired mutation(s) to a competent culture of S. thermophilus we obtained an extremely high number of specific mutants relative to unaffected wild type bacteria. Thus, the desired mutants can be identified without antibiotic selection or the use of any other marker gene. The helper plasmid is unstable and is easily cured from the host cell after the desired mutant has been isolated. It is therefore possible to introduce point mutations or other changes into the genome of bacteria such as S. thermophilus that must be regarded as food grade and should be acceptable for the consumer.

In sum, the new technique can be used to make food grade mutants of bacteria such as Streptococcus thermophilus. We believe that this technology will be of interest in industries that rely on bacterial cultures such as in for example the dairy industry since it can be used to construct new starter strains with improved properties which should be classified as GRAS (generally regarded as safe) organisms.

Thus in a first aspect the present invention provides a method of producing transformation competent bacteria, comprising at least the steps of:

(i) transforming a bacteria with a plasmid, wherein said plasmid comprises a comX gene sequence under the regulatory control of a promoter which is inducible by a transcription initiator, optionally further comprising a reporter gene under the control of a promoter;

(ii) contacting said transformed bacteria with said transcription initiator to initiate transcription of said comX gene sequence; and

(iii) optionally selecting and/or amplifying the transformation competent bacteria thus generated.

Preferably the bacteria which is transformed in step (i) is not naturally transformation competent, e.g. is not S. pneumococcus. Naturally transformation competent bacteria comprise the necessary machinery by virtue of their genetic state to be able to take up naked DNA (as described hereinafter) from the medium without the need for additional or supplementary artificial transformation techniques, such as electroporation, protoplast formation, the use of microprojectiles, CaCl₂ or heat shock methods. Bacteria that are not normally transformation competent thus do not take up naked DNA from the medium without the need for additional or supplementary artificial transformation techniques.

For example, preferred bacteria include bacteria in which early genes such as genes which produce Com W, are absent. Preferably the bacteria for use in the method has an intact ComX regulon.

Especially preferably said bacteria is S. thermophilus. Various strains are known and any suitable strain can be used. Examples of S thermophilus strains include Streptococcus thermophilus FDA strain PCI 1327 [IFO 13957] (ATCC 14485) strain LMG 18311, strain CNRZ 1066 and strain LMD-9.

As defined herein, a “transformation competent bacteria” as produced according to the above described method (or a naturally transformation competent bacteria) is a bacteria which when brought into contact with naked DNA which has a region which has considerable homology (e.g. at least 80, preferably at least 90 or 95% sequence identity) to at least a portion of the genome of the bacteria under suitable conditions for transformation incorporates said naked DNA or a portion thereof into its genome without the assistance of artificial techniques, i.e. the bacteria allows uptake of naked DNA and targeted integration of at least a portion thereof into its genome spontaneously. Transformation competent bacteria thus act in the same way as naturally transformation competent bacteria. Transformation competence may be tested as described in Example 4.

“Naked DNA” as referred to herein, refers to nucleic acid material that is not attached to or associated with other material. The DNA may be linear or circular and may be in the form of a vector construct, such as a plasmid which contains the region of homology of interest (e.g. carrying a desired mutation), optionally with flanking sequences, which will be subject to double-crossover homologous recombination in the transformation process.

The transformation process is not 100% efficient and hence a determination of whether a bacteria is transformation competent is preferably made by reference to a population of bacteria to which said naked DNA is applied. Under those circumstances “transformation competent bacteria” refers to competence induced in a significant portion of bacteria contacted with naked DNA, e.g. competence is exhibited in at least 1 in 1×10⁶ bacteria (or CFU) (i.e. 1 bacterium in every 10⁶ wild-type bacteria takes up the naked DNA and integrates at least a portion thereof into its genome), e.g. in at least 5×10⁵ or 1×10⁵ bacteria. Even higher levels of transformation may be obtained, e.g. more than 1 in 1×10⁴, 1 in 1×10³ or 1 in 1×10² bacteria. As described in the Examples high levels of transformation may be achieved by protection of the naked DNA e.g. by cloning into a plasmid to avoid nuclease action on the ends of linear DNA which may reduce transformation efficiency. Preferably the levels of competence approach those of naturally transformation competent bacteria. Bacteria which are considered incompetent or not naturally transformation competent and which are preferably used in methods of the invention exhibit competence at levels of 1 in 1×10⁷ bacteria or lower (or 1 in 1×10⁸ bacteria or lower or 1 in 1×10⁹ bacteria or lower) when contacted with naked DNA as described above.

The plasmid for use in the invention may be any plasmid suitable for transforming said bacteria. A “plasmid” encompasses any vector, isolated nucleic acid molecule, nucleic acid construct or expression vector which may be used for the transformation of the promoter:comX sequences into the bacteria which is to be rendered transformation competent and expression of the ComX protein in the first step of the method described above. For example a shuttle vector which can replicate in at least two hosts may be used. Preferably the plasmid is a multicopy plasmid.

Particularly preferred is a vector which is unstable in the bacteria, i.e. which is lost from said bacteria once transformation competence has been achieved and the subsequent transformation process to transform the bacteria with the desired DNA to generate a mutant has been achieved. Conveniently, unstable vectors which may be used exhibit stability in the presence of an exogenously added component but become unstable once the component is removed from the system, e.g. by failure to add further amounts of that component. Thus in progressive populations, e.g. within 100 generations the plasmid is substantially absent from the bacterial population. In the methods described herein, the vector pTRKH2 is employed which relies on erythromycin to retain its stability in the host. Culturing in the presence of erythromycin during the steps of transformation competence induction and transformation ensure that the plasmid is stable during these steps. Once the final transformed product has been obtained, erythromycin is not added to the culture and the plasmid loses stability and is lost from the transformed bacteria.

As such, once the transformation competent bacterium has been generated and transformed, culturing can be carried out under conditions that cause the unstable plasmid to be lost from the cells. All of the methods described herein which use an unstable plasmid thus optionally include the additional step of culturing the transformed cells under conditions that cause an unstable plasmid, when used, to be lost from the cells.

In addition to the comX gene and promoter sequences, plasmids as described herein may additionally comprise further sequences. Thus, for example, appropriate plasmids which act as expression vectors include appropriate control sequences such as for example translational (e.g. start and stop codons, ribosomal binding sites) and transcriptional control elements (e.g. promoter-operator regions, termination stop sequences) linked in matching reading frame with the other sequences of the plasmid.

Plasmids as described herein may be generated by appropriate means known in the art, particularly including methods of artificial synthesis, ligation and restriction enzyme digestion.

As used herein a “comX gene sequence” is a nucleotide sequence which encodes a ComX protein or functional part or derivative or variant thereof. A functional part of said protein is capable of performing one or more of the functions of the full length ComX protein, e.g. transcription regulation of late genes (such as comEC) involved in transformation competence in bacteria. In a preferred aspect said nucleotide sequence encodes a ComX protein from a Streptococcus species, e.g. from Streptococcus thermophilus. The complete genome of 3 different S. thermophilus strains have been sequenced and the relevant comX gene sequences from those strains may conveniently be used (Bolotin et al., 2004, Nat. Biotechnol., 22, p 1554-1558; Tettelin, 2004, Nat. Biotechnol., 22, p 1523-1524; Hols et al., 2005, FEMS Microbiol. Re., 29, p 435-463). Preferably said comX gene nucleotide sequence comprises:

(i) the nucleotide sequence:

(SEQ ID NO: 1)

atggaacaagaagtttttgttaaggcatatgaaaaggtaaggccaattgt
acttaaggcttttaggcaatactttattcagctttgggatcaagctgaca
tggagcaagaggcgatgatgactttgtatcagcttttaaaaaagtttcct
gatttagagaaagatgatgataagttacgtcgttactttaaaactaagtt
taggaatcgacttaatgatgaagtgaggcggcaggagtcagtaaaacgtc
aagctaatagacagtgctatgttgaaatttcagatattgccttttgtatt
cccaataaggagctagatatggttgatagacttgcttatgatgaacagct
taatgcatttcgtgagcagttatcatcggaagattctcttaagttggatc
gattgttgggtggtgaatgctttaggggaaggaaaaagatgatacgagag
ttaagattttggatggttgacttcgatccatgtaatgaagaagactga;
or

(ii) a portion thereof (particularly as described hereinbelow); or
(iii) a sequence which hybridizes to said sequence or portion thereof under non-stringent binding conditions of e.g. 6×SSC/50% formamide at room temperature and washing under conditions of high stringency, e.g. 2×SSC, 65° C., where SSC=0.15 M NaCl, 0.015M sodium citrate, pH 7.2; or
(iv) a sequence which exhibits at least 80%, preferably 90 or 95% e.g. at least 96, 97, 98 or 99% sequence identity to said sequence or portion thereof (as determined by, e.g. FASTA Search using GCG packages, with default values and a variable pamfactor, and gap creation penalty set at 12.0 and gap extension penalty set at 4.0 with a window of 6 nucleotides); or
(v) a sequence complementary to any of the aforesaid sequences.

The above specific comX gene sequence is from Streptococcus thermophilus LMG 18311.

Hybridizing or sequence identity related sequences (of the above sequences or sequences described hereinbelow) may be obtained by modification of the provided specific sequences, e.g. by substitution, deletion or addition and are functional equivalents as described herein. In particular “functionally-equivalent” proteins as used herein refers to proteins related to or derived from the native or naturally-occurring protein, where the amino acid sequence has been modified by single or multiple (e.g. from 2 to 10) amino acid substitutions, additions and/or deletions (e.g. N or C terminal truncation), but which nonetheless retain the same function to a lesser or greater extent than the naturally occurring molecules. Such functions are described in the definition of “portions” hereinafter. Such proteins are encoded by “functionally-equivalent nucleic acid molecules” (and may preferably include from 2 to 30 base substitutions, additions and/or deletion) which are generated by appropriate substitution, addition and/or deletion of one or more bases.

Functionally-equivalent variants mentioned above include in particular natural biological variations (e.g. allelic variants or geographical variations within a species or alternatively in different genera) and derivatives prepared using known techniques. For example, nucleic acid molecules encoding functionally-equivalent proteins may be produced by chemical synthesis or in recombinant form using the known techniques of site-directed mutagenesis including deletion, random mutagenesis, or enzymatic cleavage and/or ligation of nucleic acids.

“Portions” as referred to above in connection with nucleotide sequences, preferably comprise at least 30% of the mentioned sequence, e.g. at least 50, 70, 80 or 90% of the sequence, e.g. in connection with the comX gene a portion comprises 250 or more bases, preferably 350 or more, or 450 or more bases and encodes a sequence which is capable of performing one or more of the functions of the full length ComX protein, e.g. transcription regulation of late genes (such as comEC) involved in transformation competence in bacteria.

Portions as referred to in connection with the corresponding amino acid sequences comprise comparable lengths as those encoded by the above described nucleotide sequences, e.g. 80 or more residues, preferably more than 115 or 150 residues of the ComX protein. In relation to the bacteriocin promoter described herein, suitable portions are nucleotide sequences of at least 100, preferably at least 150, or 200 bases and which have the functional property that they are regulated and act as an inducible promoter in the plasmid as described herein. In relation to the transcription initiator described herein, suitable portions are at least 15, 20 or 25 amino acids in length and maintain the functional property that they induce the bacteriocin promoter as described herein. The existence of the desired functional properties may be determined by analysis of the portion (or of sequences with defined sequence identity) in methods of the invention, e.g. to determine if transformation competence is achieved.

Alternatively viewed, especially preferably said comX gene sequence encodes an amino acid sequence which comprises:

(i) the amino acid sequence:

(SEQ ID NO: 2)

MEQEVFVKAYEKVRPIVLKAFRQYFIQLWDQADMEQEAMMTLYQLLKKFP
DLEKDDDKLRRYFKTKFRNRLNDEVRRQESVKRQANRQCYVEISDIAFCI
PNKELDMVDRLAYDEQLNAFREQLSSEDSLKLDRLLGGECFRGRKKMIRE
LRFWMVDFDPCNEED;
or

(ii) a portion thereof (particularly as described hereinbefore); or
(iii) a sequence which exhibits at least 80%, preferably 90 or 95% e.g. at least 96, 97, 98 or 99% sequence identity to said sequence or portion thereof (as determined by, e.g. using the SWISS-PROT protein sequence databank using FASTA pep-cmp with a variable pamfactor, and gap creation penalty set at 12.0 and gap extension penalty set at 4.0, and a window of 2 amino acids).

Especially preferably said comX gene nucleotide sequence is a naturally occurring sequence or comprises a portion of a naturally occurring sequence, particularly a sequence from Streptococcus, especially preferably from S. thermophilus. In a particularly preferred embodiment, said comX gene sequence has the specific sequence set forth above or a portion thereof comprising at least 400 or 450 bases. Preferred sequences include analogous and related sequences from other species or genera.

As defined herein “under the regulatory control of a promoter” indicates that the transcription and hence expression of said comX gene is dependent on induction of the promoter.

The promoter which is inducible by a transcription initiator is a promoter which is a strong inducible promoter whose activity (i.e. regulating transcription from said promoter) is controlled directly or indirectly by the addition of the transcription initiator. As referred to herein an inducible promoter is a promoter which on addition of one or more exogenous components initiates transcription of a downstream gene. Such promoters are well known in the art. Promoters for use in the invention are strong promoters, i.e. on induction in the relevant cell (i.e. in a bacterium that is to be made transformation competent as defined above) yield high levels of transcription of the downstream gene. Examples of strong inducible promoters include promoters, from bacteria, involved in the production of bacteriocins which are regulated by a two-component system. For example nisin may be used to induce transcription from the nisin promoter.

A preferred promoter according to this embodiment is the bacteriocin promoter of S. thermophilus which, as described in the Examples may be regulated indirectly by the peptide STP. In this case STP binds to a histidine kinase which phosphorylates a cognate response regulator. Once phosphorylated the response regulator activates transcription from the bacteriocin promoter. (Homologous systems from other bacteria, particularly from Streptococcus, e.g. from other S. thermophilus strains, using homologous promoters and transcription initiators are also preferred.)

Thus in a preferred aspect the promoter comprises:

(i) the nucleotide sequence:

(SEQ ID NO: 3)

CTTCAAGGTCTAGTCCTCTCTTTTATGACGAATACTGTTTATTGAAAAAT
TGTAACATAAAGAAAACGGTTTTTCATTTTTTTATGAGTATAAAATGAGA
TTTTTTTCTGAATTTTAGAAATAATATACATTAGGAATTACCATTCGGGA
CATATAGCCACTTTTTGGGACGCTAGCTCTGATAGAGACAATTGAATGCT
ATACTAAAGATGTGATTGAGAGATCACACGATAAAAATTTTAGGAGGTAG
TTGCCATG;
or

(ii) a portion thereof (particularly as described hereinbefore); or
(iii) a sequence which hybridizes to said sequence or portion thereof under non-stringent binding conditions of e.g. 6×SSC/50% formamide at room temperature and washing under conditions of high stringency, e.g. 2×SSC, 65° C., where SSC=0.15 M NaCl, 0.015M sodium citrate, pH 7.2; or
(iv) a sequence which exhibits at least 80%, preferably 90 or 95% e.g. at least 96, 97, 98 or 99% sequence identity to said sequence or portion thereof; or
(v) a sequence complementary to any of the aforesaid sequences.

The transcription initiator may act directly or indirectly on the promoter to induce transcription of the gene downstream of the promoter. Thus the transcription initiator may bind directly to the promoter, or to a molecule associated with said promoter, to induce its activity and initiate transcription or may be part of a regulatory system which induces the activity of the promoter. In the latter case, the transcription initiator may be part of a signal transduction pathway which activates one or more intermediate components wherein induction of the promoter and activation of transcription is mediated by a secondary molecule which is present in the system.

In a preferred embodiment, as described herein, a two-component regulatory system such as the systems involved in bacteriocin-production in bacteria is used. In the embodiment described in the Examples, a peptide is used which produces a cascade of events culminating in the production of a phosphorylated response regulator which activates transcription from the bacteriocin promoter.

Thus in a preferred aspect, the transcription initiator for use with a promoter as described hereinbefore (i.e. the bacteriocin promoter) comprises:

(i) the amino acid sequence:

| | |
|---|---|
| SGWMDYINGFLKGFGGQRTLPTKDYNIPQV; | (SEQ ID NO: 4) |
| or | |

(ii) a portion thereof (as described hereinbefore); or
(iii) a sequence which exhibits at least 80%, preferably 90 or 95% e.g. at least 96, 97, 98 or 99% sequence identity to said sequence or portion thereof (as determined by, e.g. using the SWISS-PROT protein sequence databank using FASTA pep-cmp with a variable pamfactor, and gap creation penalty set at 12.0 and gap extension penalty set at 4.0, and a window of 2 amino acids).

The transcription initiator may also comprise non-naturally occurring amino acids to replace the corresponding amino acids described above providing they provide the correct functionality. Such modifications are encompassed within the transcription initiators described herein.

Larger sequences may be used, in which flanking sequences are present, e.g. the native precursor of the above described peptide may be employed which has the sequence:

(SEQ ID NO: 5)

MANNTINNFETLDNHALEQVVGGSGWMDYINGFLKGFGGQRTLPTKDYNI
PQV,

or portions or sequences with levels of sequence identity as defined above.

As described above, the plasmid may further comprise a reporter gene which is under the control of a promoter. The promoter controlling the reporter gene's transcription may the same or different to the promoter which controls transcription of the comX gene. As defined herein a “reporter gene” refers to a nucleotide sequence which is capable of direct or indirect detection by the generation or presence of a signal. The signal may be any detectable physical characteristic such as conferred by radiation emission, scattering or absorption properties, magnetic properties, or other physical properties such as charge, size or binding properties of existing molecules (e.g. labels) or molecules which may be generated (e.g. colour change etc.). Techniques are preferred which allow signal amplification, e.g. which produce multiple signal events from a single reporter, e.g. by the catalytic action of enzymes to produce multiple detectable products.

Conveniently the reporter gene may be, or carry a label which itself provides a detectable signal, such as a radiolabel, chemical label, for example chromophores or fluorophores (e.g. dyes such as fluorescein and rhodamine), or reagents of high electron density such as ferritin, haemocyanin or colloidal gold. In such cases direct detection of the reporter gene may be possible. Preferred labels for use according to the invention are chromophores and fluorophores.

Preferably however indirect detection may be achieved, e.g. by expression of the product of the reporter gene wherein the product may be detected directly or indirectly. Thus for example the reporter gene may encode a protein such as an enzyme which interacts with a suitable endogenous or exogenous substrate to produce a signal such as light emission, colour change or the production of otherwise detectable products. Alternatively the reporter gene's expressed product may be detected by appropriately labelled binding partners such as antibodies to that product. A suitable reporter gene is as described in the Examples, namely the luciferase gene, the expressed product of which causes measurable luminescence on addition of D-luciferin.

Preferably the reporter gene is under the control of a promoter which is induced as a result of expression of the comX gene, i.e. to confirm transformation of the plasmid and expression of the comX gene. A suitable promoter in this regard is the late gene comEC promoter (as described in the Examples) which is activated by ComX.

In a particularly preferred aspect, the invention provides a method of producing transformation competent S. thermophilus bacteria, comprising at least the steps of:

(i) transforming a S. thermophilus bacteria with a plasmid, wherein said plasmid comprises a comX gene sequence which comprises:
(a) the nucleotide sequence:

(SEQ ID NO: 1)

atggaacaagaagtttttgttaaggcatatgaaaaggtaaggccaattgt
acttaaggcttttaggcaatactttattcagctttgggatcaagctgaca
tggagcaagaggcgatgatgactttgtatcagcttttaaaaaagtttcct
gatttagagaaagatgatgataagttacgtcgttactttaaaactaagtt
taggaatcgacttaatgatgaagtgaggcggcaggagtcagtaaaacgtc
aagctaatagacagtgctatgttgaaatttcagatattgccttttgtatt
cccaataaggagctagatatggttgatagacttgcttatgatgaacagct
taatgcatttcgtgagcagttatcatcggaagattctcttaagttggatc
gattgttgggtggtgaatgctttaggggaaggaaaaagatgatacgagag
ttaagattttggatggttgacttcgatccatgtaatgaagaagactga;
or

(b) a portion thereof; or
(c) a sequence which hybridizes to said sequence or portion thereof under non-stringent binding conditions and washing under conditions of high stringency; or
(d) a sequence which exhibits at least 80% sequence identity to said sequence or portion thereof; or
(e) a sequence complementary to an of the aforesaid sequences;
   under the regulatory control of a promoter comprising:
(a) the nucleotide sequence:

(SEQ ID NO: 2)

CTTCAAGGTCTAGTCCTCTCTTTTATGACGAATACTGTTTATTGAAAAAT
TGTAACATAAAGAAAACGGTTTTTCATTTTTTTATGAGTATAAAATGAGA
TTTTTTTCTGAATTTTAGAAATAATATACATTAGGAATTACCATTCGGGA
CATATAGCCACTTTTTGGGACGCTAGCTCTGATAGAGACAATTGAATGCT
ATACTAAAGATGTGATTGAGAGATCACACGATAAAAATTTTAGGAGGTAG
TTGCCATG;
or

(b) a portion thereof; or
(c) a sequence which hybridizes to said sequence or portion thereof under non-stringent binding conditions and washing under conditions of high stringency; or
(d) a sequence which exhibits at least 80% sequence identity to said sequence or portion thereof; or
(e) a sequence complementary to any of the aforesaid sequences;
   which is inducible by a transcription initiator comprising:
(a) the amino acid sequence:

| | |
|---|---|
| SGWMDYINGFLKGFGGQRTLPTKDYNIPQV; | (SEQ ID NO: 4) |

(b) a portion thereof; or
(c) a sequence which exhibits at least 80% sequence identity to said sequence or portion thereof,
   optionally further comprising a reporter gene under the control of said promoter;
   (ii) contacting said transformed bacteria with said transcription initiator to initiate transcription of said comX gene sequence; and
   (iii) optionally selecting and/or amplifying the transformation competent S. thermophilus bacteria thus generated.

Preferably the plasmid for use in the methods of the invention is as described in the Examples (i.e. the pXL plasmid) or a plasmid having at least 80, 90, 95, 96, 97, 98 or 99% sequence identity to said plasmid or having said homology to at least the portions comprising the promoter and comX gene sequence.

Plasmids as described herein form further aspects of the invention. Isolated nucleic acid molecules comprising the comX gene sequence as described herein under the regulatory control of a promoter as described herein form further aspects of the invention.

In performing the method, the initial transformation step is performed using any convenient artificial means (as described above). Conveniently, transformation is achieved by electroporation. Suitably transformed clones may be selected e.g. by analysis of the clones for the presence of nucleotide sequences present in the plasmid. The transformed bacteria may then be brought into contact with the transcription initiator, by the addition of the initiator into the bacteria's media. To determine if transformation competence has been achieved, signals generated by the reporter gene may be analyzed. Selection of transformation competent bacteria may be achieved by selection of clones exhibiting activity from the reporter gene and these bacteria may be amplified by continued growth.

In a further aspect of the invention, there is provided a method of producing transformed bacteria comprising a plasmid as described herein wherein said method comprises transforming said bacteria with said plasmid. Transformed bacteria thus produced and methods of producing transformation competent bacteria involving the additional step of contacting said transformed bacteria with a transcription initiator as described herein form further aspects of the invention.

The invention further extends to transformation competent bacteria produced according to the above described method.

Once transformation competent bacteria have been generated these may be used to produce mutant bacteria strains by transformation.

Thus in a further aspect, the present invention provides a method of producing a mutant bacteria (preferably a mutant S. thermophilus bacteria) comprising at least the steps of:

(i) contacting a transformation competent bacteria prepared according to the above-described methods with homologous DNA comprising a mutation under conditions to allow transformation of said bacteria with said homologous DNA.

Homologous DNA as described herein refers to DNA which contains regions of sufficient homology to allow double-crossover homologous recombination into the genome of the bacteria into which the DNA is transformed. The homologous DNA has previously been referred to herein as “naked DNA” and these terms are used interchangeably. Thus the DNA may be in a linear or circular form and sequences additional to the region of homology may be provided. For example flanking sequences may be present to mediate targeted integration of the mutated region into the bacterial genome. To aid transformation efficiency, the terminal ends of the DNA for transformation are preferably protected from nuclease activity, e.g. by cloning into a plasmid or use of a circular vector or construct. The presence of flanking sequences at one or both ends of the homologous region can also be used to stabilise the DNA for transformation against nuclease activity.

As mentioned above, the homologous DNA contains a region of homology for recombination. Preferably said region of homology is in the order of 100 bp-2 kbp in length, especially preferably 300-1000 bp, 500-3000 bp or 1500-2500 bp in length. Preferably the DNA in the region of homology has at least 90% sequence identity (e.g. at least 92, 94, 96, 97, 98 or 99% sequence identity) to the corresponding region in the genome of the bacteria into which transformation is to be performed. Thus said region of homology may contain one or more mutations in the sequence, e.g. addition, deletion or substitution of from 1 to 20 bases, preferably 1, 2, 3, 4, 5, 6, 7, 8, 9 or 10 bases. The mutations are preferably present in the centre of the homologous region, e.g. in the central 20, 40, 60, 80, 100, 150, 200, 250, 500, 750, 1000 base pairs of the homologous region.

Suitable conditions to achieve transformation are known in the art and may be adjusted to optimize the transformation process. Since the bacteria into which the mutant DNA to be introduced are transformation competent, only simple methods of transformation in which the homologous DNA is brought into contact with the bacteria are required. In one embodiment, the transformation competent bacteria are exposed to the homologous DNA for approximately 2 hours (e.g. about 1.5-about 2.5 hours, about 1 to about 3 hours, or about 0.5 to about 3.5 hours).

The concentration of the DNA is in general in the region of 1 μg per ml of competent cells (e.g. 0.01-100 μg per ml, 0.1-10 μg/ml or 0.5-5 μg/ml) when the concentration of the cells is such that the OD₅₅₀ is about 0.5. Conveniently, conditions described in the Examples may be used.

Preferably, one or more of the steps of the methods of the invention, or the method in its entirety, is performed in growth medium that comprises one or more of heart infusion, neopeptone (or peptonen or peptone e.g. casein or yeast peptone), dextrose, sodium chloride, disodium phosphate and sodium carbonate.

The heart infusion can e.g. be from beef heart and this can be present in a range of from about 0.5 to about 10 g per litre, preferably about 1.1 to about 5.1 g/l, more preferably about 2.1 to about 4.1 g/l, and even more preferably about 2.6 to about 3.6 g/l. Approximately 3.1 g/l is highly preferred.

The neopeptone (or peptonen or peptone e.g. casein or yeast peptone) can be present in a range of from about 10 to about 50 g per litre, preferably about 15 to about 30 or about 25 g/l, more preferably about 17.5 to about 22.5 g/l, and even more preferably about 20 g/l.

The dextrose and the sodium chloride can each independently be present in a range of from about 0.5 to about 10 g per litre, preferably about 1.0 to about 5.0 g/l, more preferably about 1.5 to about 3 g/l, and even more preferably about 1.75 to about 2.25 g/l. Approximately 2 g/l is highly preferred.

The disodium phosphate can be present in a range of from about 0.1 to about 2 g per litre, preferably about 0.2 to about 1 g/l, more preferably about 0.3 to about 0.8 g/l, and even more preferably about 0.3 to about 0.5 or 0.6 g/l. Approximately 0.4 g/l is highly preferred.

The sodium carbonate may be present in a range of from about 0.5 to about 10 g per litre, preferably about 1.0 to about 5.0 g/l, more preferably about 1.5 to about 3 g/l, and even more preferably about 2.0 to about 2.75 g/l. Approximately 2.5 g/l is highly preferred.

Todd-Hewitt broth (Todd, E. W., et al. J. Pathol. Bacteriol. 35, 1-97, (1932)) which is widely available (e.g. from Difco Laboratories) is preferred.

The composition of Todd-Hewitt broth is (in g/l)

| | |
|---|---|
| Beef Heart, Infusion | 3.1 |
| Casein/Yeast Peptone, neopeptone or peptonen | 20.0 |
| Sodium Chloride | 2.0 |
| Disodium Phosphate | 0.4 |
| Sodium Carbonate | 2.5 |
| Dextrose | 2.0 |

The pH is in general 7.8 +/− 0.2 at 25° C.

The growth medium may optionally be further supplemented, e.g. with glucose e.g. at about 0.1 to about 10%, about 0.2 to about 5% or about 0.5 to about 2% or about 1%, preferably 0.8%.

Following the transformation step, the method may additionally comprise the steps of selecting and/or amplifying the mutant bacteria thus generated. This may be achieved for example by using labelled oligonucleotide probes directed to the mutant sequences and subsequent growth of the selected colonies.

As discussed above, once a mutant bacteria has been obtained, any unstable plasmid present in said bacteria can optionally be removed by culturing said bacteria under conditions that allow the unstable plasmid to be lost from the cell. This is known as “curing”. The growth conditions are altered such that the unstable plasmid is lost from the cell.

Mutant bacteria produced according to the above described method form further aspects of the invention.

Mutant bacterial strains produced according to the methods described herein have a variety of uses. Principally however they may be used as starter cultures in the production of food products.

Thus a further aspect of the invention provides a method of producing a food product comprising at least the step of fermentation using a mutant bacteria produced according to the method described herein. Thus for example said method may comprise the production of cheese or yoghurt in which milk is fermented with a mutant bacteria of the invention. Especially preferably said mutant bacteria are mutants of S. thermophilus, particularly mutants of strain LMG 18311.

Food products generated by the above described methods form further aspects of the invention.

The following Examples are given by way of illustration only in which the Figures referred to are as follows:

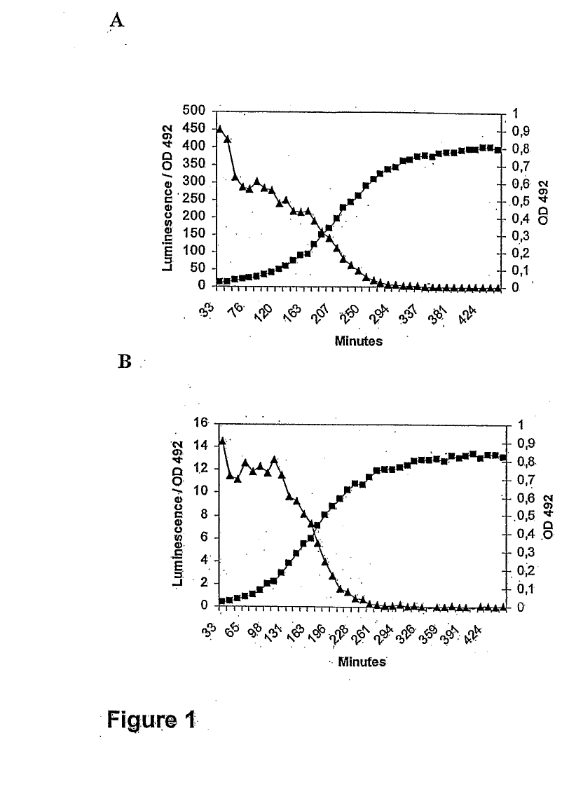

FIG. 1 shows expression of the luc reporter gene (▴) during growth of S. thermophilus LMG 18311 from logarithmic to stationary phase. (▪) Growth curve of culture. (a) Expression of luciferase driven by the comX promoter. (b) Expression of luciferase driven by the comEA late gene promoter;

FIG. 2 shows the effect of the growth medium on STP induced overexpression of the luciferase reporter protein. Expression of the luc reporter gene is driven by the promoter of the putative bacteriocin gene stbD. The S. thermophilus BP strain was grown in THG (black curves) or HJGL (grey curves) from logarithmic to stationary phase. The STP peptide pheromone (curves with circles) was added at time zero; and

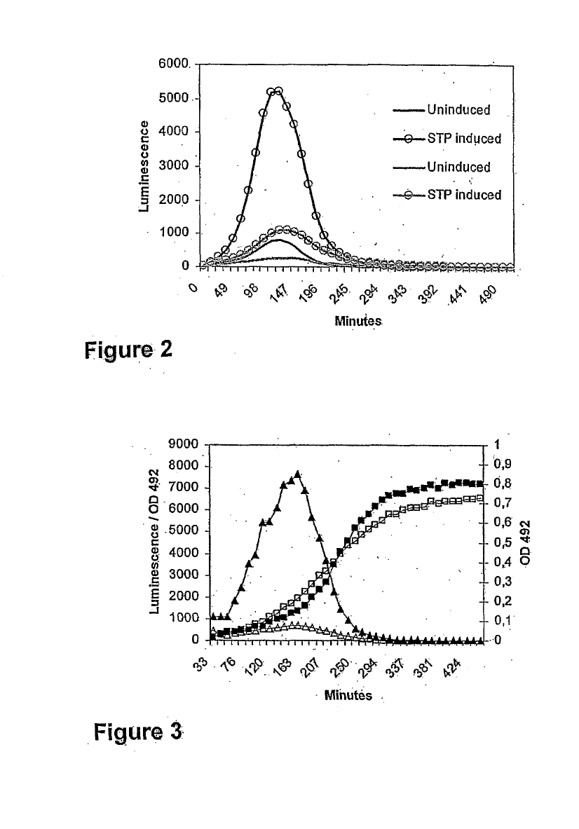

FIG. 3 shows the transcriptional activation of late competence genes by STP induced overexpression of ComX in S. thermophilus LMG 18311 carrying the pXL plasmid. ComX activates transcription from late gene promoters and the curves show expression of late competence genes in STP (▴) and uninduced (Δ) S. thermophilus cultures. (▪) Growth curve of STP induced culture. (□) Growth curve of uninduced culture. The STP peptide pheromone was added at time zero.

### EXAMPLES

### Methods

### Bacterial Strains and Growth Media

S. thermophilus strains LMG 18311 (ATCC no. BAA-250) and LMD-9 (ATCC no. BAA-491) were cultivated in Todd-Hewitt broth (Difco Laboratories) supplemented with 0.8% glucose (THG) or Hogg-Jago glucose broth (HJG) consisting of 3% tryptone, 1% yeast extract, 0.2% beef extract, 0.5% KH₂PO₄, and 0.5% glucose. HJGL is Hogg-Jago glucose broth supplemented with 0.5% lactose, whereas HJGLS is HJGL supplemented with 0.4M D-sorbitol. Agar plates were prepared by adding 1.5% (w/v) agar to the media.

### Construction of Plasmids.

The three reporter plasmids pXP, pEAP and pBP were constructed by fusing the comX, comEC and stbD promoters to the firefly luciferase gene and ligating the resulting fragments into the pTRKH2 shuttle vector (O'Sullivan & Klaenhammer, 1993, Gene, 137, p 227-231). Briefly, the luciferase gene was amplified in three separate PCR reactions using the primer pairs LXP/LR (pXP), LCB/LR (pEAP), and LBP/LR (pBP), and a plasmid (pR424) carrying the luc gene as template (Chastanet et al., 2001, J. Bacteriol., 183, p 7295-7307). Similarly, PCR with the primer pairs CXPF/CXPL, CBF/CBL, and BPF/BPL, and genomic DNA from S. thermophilus LMG 18311, were used to amplify fragments corresponding to the comX, comEC and stbD promoters, respectively. Then, promoter and luc gene fragments with complementary overlapping ends were combined and amplified in PCR reactions containing the appropriate external primers. The primer pairs CXPF/LR, CBF/LR and BPF/LR were used to generate the XL, EAP, and BP fragments, respectively. Finally, the three fragments' were cloned into the pCR 2.1-TOPO vector (Invitrogen), excised by XhoI and PstI, and ligated into the corresponding sites of the pTRKH2 vector (O'Sullivan & Klaenhammer, 1993, supra). The resulting reporter plasmids pXP, pEAP and pBP were electroporated into S. thermophilus LMG 18311 as described below, giving rise to the XP, EAP and BP strains.

To construct the pXL plasmid, a DNA fragment containing the comX gene joined to the promoter of a putative bacteriocin gene (stbD), was ligated into the pEAP plasmid (see above). The fragments corresponding to the bacteriocin promoter (P_{stbD}) and the comX gene were amplified from S. thermophilus LMG 18311 DNA, using the primers P1/P2 and X1/X2, respectively. The P2 and X1 primers contains NcoI sites at their 5′-ends coinciding with the start codon of the comX gene. Next, the P_{stbD} and comX fragments were cloned separately into the pDrive vector (Qiagen). The comX fragment was excised from pDrive by digestion with NcoI and XbaI, and ligated into the corresponding sites of the pDrive vector carrying the stbD promoter fragment. Then the joined P_{stbD}:: comX fragment was excised from the pDrive vector by digestion with PstI and SacI, and ligated into pTRKH2 precleaved with the same enzymes. Finally, the P_{stbD}:: comX fragment was excised from pTRKH2 by digestion with BamHI and EcoRV and ligated into pEAP precleaved with BamHI and SmaI. The resulting construct, pXL, contains an expression module (P_{stbD}:: comX) and a reporter module (P_{comEC}:: luc) inserted in opposite orientations. All PCR reactions described above were carried out with the Phusion™ High-Fidelity DNA Polymerase (Finnzymes).

### Primers

| | | SEQ |
|---|---|---|
| Name | Sequence | ID NO |
| CBF | AGTGTAACTGCAGAATACTTGCAGGTCTATCGATCG | 6 |
| | AT | |
| CBL | TTTGGCGGATCTCATAAGGACCTCCTCATAAACCTAT | 7 |
| | TC | |
| CXPF | CGCTTTCTGCAGCTATCACTCTAATACAATCCTGTGG | 8 |
| | AA | |
| CXPL | TTGGCGGATCTCATTGAACCTCCAATAATAAATATAA | 9 |
| | ATTCTGT | |
| BPF | GTAAATCTGCAGCTTCAAGGTCTAGTCCTCTCT | 10 |
| BPL | TTGGCGGATCTCATGGCAACTACCTCCTAAAATTTTT | 11 |
| | ATC | |
| LCB | TATGAGGAGGTCCTTATGAGATCCGCCAAAAACAT | 12 |
| LR | CATATGGCTCGAGTGCACTCTCAGTACAATCTGCTC | 13 |
| LXP | TATTGGAGGTTCAATGAGATCCGCCAAAAACATAAAG | 14 |
| | AAAGGC | |
| LBP | TAGGAGGTAGTTGCCATGAGATCCGCCAAAAACATAA | 15 |
| | AGAAAGGC | |
| P1 | GTTTGAGTTGCCATGGCAACTACCTCC | 16 |
| P2 | ATTAGGATCCTTCAAGGTCTAGTCCTCTCTTTTATGA | 17 |
| | CG | |
| X1 | ATTATCTAGACCAAGAATTACTGGAAACACAATAGA | 18 |
| | GG | |
| X2 | GGAGGTTCCATGGAACAAGAAGTTTTTGTTAAGGC | 19 |
| EC1 | GAGGCATCATTGGAAGAATAGAGCAGC | 20 |
| EC2 | AAGCTTAAGATCTAGAGCTCGAGGATCAAAAACTAGA | 21 |
| | GAGAAGATTGCCGTCAG | |
| EC3 | AGCATGCATATGCATCCGGAGTCCTAGCTTGTTTCAG | 22 |
| | TTTGTCTCAATG | |
| EC4 | CCATCCCTTAAACCGAATGGCACC | 23 |
| Kana-F | ATCCTCGAGCTCTAGATCTTAAGCTT | 24 |
| Kana-R | ACTCCGGATGCATATGCATGCT | 25 |

Preparation of Electrocompetent S. thermophilus LMG 18311 Cells.

An overnight culture grown at 37° C. was diluted 100-fold in preheated HJG (37° C.) and incubated until it reached OD₆₆₀=0.3. The culture (50 ml) was then diluted 1:1 in pre-warmed HJG containing 20% glycine. After incubation at 37° C. for 1 hour cells were harvested by centrifugation (4000×g for 10 min at 4° C.), and washed twice with one volume of ice-cold electroporation buffer (5 mM KHPO₄; 0.4 M D-sorbitol; 10% glycerol; pH 4.5). Finally, pelleted cells were resuspended in 4 ml ice-cold electroporation buffer, divided into aliquots, and frozen on an ethanol-dry ice bath. Aliquoted electrocompetent S. thermophilus LMG 18311 cells were stored at −80° C.

### Electroporation

A Bio-Rad MicroPulser unit was used to transform S. thermophilus LMG 18311 cells by electroporation. Competent cells were thawed on ice and 80 μl cell suspension was mixed with 1 μg recombinant pTRKH2 plasmid DNA. After 30 min on ice, the cells were transferred to an electroporation-cuvette with a 0.1-cm gap between the electrodes. A single pulse of 1.6 kV lasting 2.5 ms was delivered. The electroporated cells were immediately resuspended in 1 ml ice-cold HJGLS and incubated for 3 h at 37° C., before spreading on HJGL plates containing 2 μg/ml erythromycin. Transformants were picked following 24-48 h incubation at 37° C. Isolated clones were verified by PCR using primers (M13F and M13R) that are complementary to sequences flanking the multiple cloning site of the pTRKH2 plasmid.

### Luciferase Reporter Assay

Detection of luciferase activity was performed essentially as previously described by Chastanet et al. (2001, supra). Strains were grown in THG to OD₅₅₀=0.4, aliquoted, and maintained as glycerol stocks at −80° C. Shortly before use, glycerol stocks were thawed and diluted ten times in THG. For each test sample, 280 μl diluted culture was mixed with 20 μl of firefly D-luciferin (10 mM solution in THG) and transferred into a 96-well Corning NBS plate with a clear bottom. If appropriate the peptide pheromone STP was added to a final concentration of 250 ng/ml immediately before starting the experiment. The plate was incubated at 37° C. in an Anthos Lucy 1 luminometer for 7.5 hours. Optical density (OD₄₉₂) and luminescence were measured automatically by the luminometer at 10-minute intervals.

Natural Transformation of S. thermophilus LMG 18311

S. thermophilus LMG 18311 cells harbouring pXL were grown overnight at 37° C. in Todd-Hewitt broth (Difco Laboratories) supplemented with 0.8% glucose and 2 μg/ml erythromycin. The next day the culture was diluted to OD₅₅₀=0.5 in the same medium prewarmed to 37° C. Then, 1 ml diluted culture was transferred to a 1.5 ml Eppendorf tube containing 250 ng STP, and the sample was placed in a water bath at 37° C. Two hours later transforming DNA was added, and the incubation was continued for an additional two hours. Finally, the sample was put on ice, serially diluted, and spread on HJGL agar plates containing the appropriate antibiotic (50 μg/ml streptomycin or 100 μg/ml kanamycin). To avoid loosing the pXL plasmid 2 μg/ml erythromycin must be added to the HJGL agar plates. Curing of the pXL plasmid was obtained by cultivating transformants in antibiotic-free medium for about 100 generations.

### Disruption of the ComEC Gene

The comEC gene disruption cassette consists of a kanamycin resistance gene flanked by two 800-1000 bp DNA fragments corresponding to the 5′ and 3′ regions of the comEC gene. In a first step, the kanamycin resistance gene was amplified by PCR from the pFW13 vector (Podbielski et al., 1996, Gene, 177, p 137-147) using the primers Kana-F and Kana-R. In a second step, the 5′ and 3′ flanking fragments were generated in two separate PCR-reactions with the primer pairs EC1/EC2 and EC3/EC4, and genomic DNA from S. thermophilus LMG 18311 as template. The EC2 and EC3 primers used to amplify the flanking sequences contain 22 base pairs extensions homologous to the 5′ and 3′ ends of the kanamycin gene, respectively. After agarose gel purification of all PCR fragments, the kanamycin resistance gene was first joined to the 5′ flanking fragment in a PCR-reaction containing the two DNA fragments and the EC1 and Kana-R primers. In the same way, the kanamycin resistance gene was joined to the 3′ flanking fragment in a PCR reaction containing both fragments and the Kana-F and EC4 primers. Finally, the two combined fragments were joined in a PCR reaction containing the EC1 and EC4 primers. The resulting comEC gene disruption cassette was purified by a PCR purification kit from Qiagen and used directly to transform competent S. thermophilus LMG 18311 cells carrying the pXL plasmid. In addition the gene disruption cassette was cloned into the pCR 2.1-TOPO vector (Invitrogen), according to the manufacturer's instructions.

### Example 1

### ComX is Expressed in S. thermophilus During Early Logarithmic Growth

Natural transformation is a highly efficient tool for genetic manipulation that has been used successfully in S. pneumoniae for more than sixty years. Experiments were conducted to determine whether S. thermophilus is a naturally transformable species. We chose to work on S. thermophilus LMG 18311, which has been isolated from yoghurt produced in the United Kingdom in 1974. Initially, experiments were carried out to establish if transformants could be obtained by adding homologous genomic DNA containing a streptomycin resistance marker to LMG 18311 cultures grown under various conditions. All results were negative suggesting that ComX and/or the late genes are not expressed under the conditions used.

To be able to monitor the activity of the comX promoter in a growing culture of LMG 18311 cells over time and under various conditions, we used the shuttle plasmid pTRKH2 to construct a reporter plasmid, pXP, harbouring a transcriptional fusion between the comX promoter and the firefly luciferase gene. The pXP plasmid was subsequently introduced into S. thermophilus LMG 18311 by electroporation giving rise to the XP strain. Luciferase activity was monitored by growing cultures of the XP strain at 37° C. in a Lucy 1 luminometer (Anthos) in 96 well microtiter plates with clear bottoms (Corning) essentially as described previously (Chastanet et al., 2001, supra). Both optical density (OD₄₉₂) as well as light production was measured automatically by the luminometer at 10-minute intervals. Unexpectedly, we discovered that the comX promoter is active during early to approximately mid-logarithmic phase in XP cells grown in THG medium at 37° C. As the culture approached stationary phase the activity of the comX promoter declined to zero (FIG. 1A).

### Example 2

### Low Level Expression of Late Genes During Early Logarithmic Phase

Even though ComX is expressed in early logarithmic phase, we were not able to obtain transformants when cultures at this stage of growth were subjected to purified genomic DNA from a streptomycin resistant mutant of strain LMG 18311. A possible explanation for this negative result could be undetected loss-of-function mutations in the transformation machinery. Alternatively, the level of ComX produced might be too low to significantly activate expression of the late genes. To determine whether this could be the case we constructed a plasmid similar to pXP, except that we exchanged the comX promoter with the promoter of the late gene comEA (stu1562). The resulting plasmid, pEAP, was electroporated into S. thermophilus LMG 18311, giving rise to the EAP strain. The activity of the comEA promoter was monitored by growing the EAP strain in the Lucy 1 luminometer exactly as described for the XP strain above. The data obtained revealed a very small peak of luminescence roughly coinciding with the peak representing the activity of the comX promoter (FIGS. 1A and B). From the reporter assay alone, it is not possible to know whether the comEA promoter operates at a very low level in all bacteria in the culture, or if it is highly expressed in just a tiny fraction of the cells. In any case, the results indicate that the level of ComX produced is too low to turn on the competent state in a significant fraction of the bacterial population.

### Example 3

### Development of an Inducible System for High-Level Expression of ComX

In S. pneumoniae Morrison and coworkers have shown that a product of the early genes, termed ComW, is needed in addition to ComX for efficient competence induction (Chastanet et al., 2001, supra; Luo et al., 2003, Mol. Microbiol., 50, p 623-633). Evidence indicates that ComW contributes to the stabilization of ComX against proteolysis, and that it in addition might be required for full activity of the sigma factor (Luo & Morrison, 2004, Mol. Microbiol., 54, p 172-183; Sung & Morrison, 2005, J. Bacteriol., 187, p 3052-3061). We were not able to identify any homologue of ComW in S. thermophilus, but found it reasonable to assume that ComX is unstable also in this species.

We hypothesized that it might be possible to induce competence in S. thermophilus if sufficiently high levels of ComX could be obtained. Using a strong constitutive promoter was not considered appropriate as constant high levels of ComX would interfere with the normal transcription pattern of the cell. We therefore selected an inducible system with a strong promoter to drive expression of ComX. Such systems have not been developed for S. thermophilus, and we therefore set out to identify strong promoters in the genome of LMG 18311 that could be controlled by exogenously added inducer molecules. Bacteriocin promoters are good candidates as these antimicrobial peptides are usually highly expressed. In addition, bacteriocin production in lactic acid bacteria is often regulated by a quorum-sensing mechanism (Mathiesen et al., 2004, Lett. Appl. Microbiol., 39, p 137-143). A locus encoding proteins with high homology to the pneumococcal BlpABCHR quorum-sensing system was identified in Streptococcus thermophilus LMG 18311. The BlpABCHR system regulates bacteriocin production in Streptococcus pneumoniae by monitoring the extracellular concentration of a peptide pheromone encoded by blpC (de Saizieu et al., 2000, J. Bacteriol., 182, p 4696-4703; Reichmann & Hakenbeck, 2000, FEMS Microbiol. Lett., 190, p 231-236). The homologous system in S. thermophilus, termed StbABCHR, contains a corresponding gene stbC (stu1688) encoding a possible peptide pheromone (STP) that presumably controls bacteriocin production in S. thermophilus.

We synthesized this peptide (NH₂—SGWMDYINGFLKGFGGQRTLPTKDYNIP QV-COOH) and found that it activates transcription of a luc reporter gene placed behind the promoter of the bacteriocin-like gene stbD (stu1685). The reporter construct (pBP) was made in the same way as pXP and pEAP, except that the stbD promoter was inserted upstream the luciferase gene. We tested this STP inducible expression system in different media and found that the level of luminescence obtained was by far the highest in THG-medium (FIG. 2).

### Example 4

### Overexpression of ComX Induces the Competent State in S. thermophilus

To determine whether the new expression system could drive ComX production to the level required for activating transcription of the late genes, a new plasmid based on pEAP was made. This plasmid (pXL) was constructed by ligating a DNA fragment, consisting of a transcriptional fusion between the stbD promoter and the comX gene, into unique restriction sites of the pEAP plasmid. To avoid transcriptional read-through the expression and reporter modules of the pXL plasmid were inserted in opposite directions. The resulting construct was introduced into the LMG 18311 strain by electroporation. The effect of STP induced overproduction of ComX on late gene expression was subsequently monitored by measuring light emission from XL cells in the Lucy 1 luminometer. The results showed that a culture of XL cells subjected to 250 ng/ml of STP displayed an approximately 600 fold increase in luminescence compared to a corresponding culture of bacteria harbouring the pEAP plasmid (FIGS. 1B and 3). No effect on luciferase expression was seen when cultures of the EAP and XP strains were treated with the STP peptide pheromone, demonstrating that the dramatic increase in light production observed with the XL strain must be due to STP induced overexpression of ComX. Our results also revealed that ComX is expressed in uninduced XL cells due to a leaky stbD promoter However, peak luminescence of cultures treated with STP was about seven-fold higher than the luminescence of uninduced cultures (FIG. 3). We also discovered that in the absence of a selection pressure the pXL plasmid is rapidly lost from its host. Presumably the presence of ComX, expressed from the leaky stbD promoter, disturbs the normal functions of the bacterial cell.

Having constructed an inducible ComX expression system that efficiently activates transcription from late gene promoters, we sought to determine whether the transformation machinery of S. thermophilus LMG 18311 was functional. To our delight 3×10³ (SE±0.9×10³; n=4) streptomycin resistant colony forming units (CFUs) per ml of culture were obtained when homologous genomic DNA carrying a streptomycin marker was added to cultures of the XL strain pretreated with the STP pheromone (see Methods for experimental details). The total number of CFUs in the culture was estimated in parallel and was determined to be 5×10⁸ (SE±1×10⁸; n=4).

To demonstrate that natural genetic transformation is a very efficient tool for genetic manipulations in S. thermophilus, we decided to make a comEC knockout mutant. ComEC, also called CelB, has been shown to constitute a key component of the DNA uptake apparatus in Bacillus subtilis, Streptococcus pneumoniae and other genetically transformable bacteria (Peterson et al., 2004, supra; Draskovic & Dubnau, 2005, Mol. Microbiol., 55, p 881-896). Thus, if the comEC knockout mutant displays a competence negative phenotype, it would prove that this mutant, and other mutants made with the same technique, was generated by natural transformation.

Thus, to ensure that the observed acquisition of streptomycin resistance had taken place by natural transformation we decided to check whether the process depends on a functional comEC gene. The gene encoding ComEC is located on the same transcriptional unit as comEA. To disrupt the comEC gene we used PCR to generate a DNA fragment consisting of a kanamycin marker fused to ˜1000 bp flanking regions amplified from the 5′ and 3′ halves of the comEC gene of S. thermophilus. This fragment was added to a STP induced culture of the XL strain at a concentration of 1 μg/ml. After 2 hours at 37° C. the bacteria were spread on agar plates containing 100 μg/ml of kanamycin and further incubated at 37° C. for 18-24 hours. We obtained 7×10³ (SE±1×10³; n=4) CFUs per ml on the agar plates containing kanamycin, and ˜5×10⁸ CFUs per ml on the control plates lacking the antibiotic. Correct integration of the gene disruption cassette into the comEC gene by double-crossover homologous recombination was verified by PCR in ten randomly picked kanamycin resistant colonies.

Next, we tested the transformability of the XL ΔcomEC strain using genomic DNA from the streptomycin resistant LMG 18311 mutant as a selectable marker. No transformants were obtained, demonstrating that the XL strain becomes non-competent when the comEC gene is disrupted.

When performing transformation with a linear DNA fragment, such as the comEC gene-disruption cassette described above, the ends of the fragments may be attacked and shortened by nucleases resulting in reduced transformation efficiency. In an attempt to further increase the transformation efficiency we protected the ends of the comEC gene-disruption cassette by cloning it into the pCR2.1-TOPO plasmid (Invitrogen). By using this strategy we obtained 3×10⁶ (SE±0.4×10⁶; n=4) kanamycin resistant CFUs per ml when 3 μg/ml of plasmid DNA was added to STP induced cultures of the XL strain. The total number of CFUs per ml of competent culture was estimated to be ˜5×10⁸. As described above, correct integration of the comEC gene-disruption cassette was verified by PCR in ten randomly picked colonies. These results show that approximately 1% of the streptococcal chains, present in the competent culture receiving 3 μg/ml of recombinant plasmid DNA, will give rise to a colony when cultivated on agar plates containing kanamycin.

### Example 5

### Introduction of Point Mutations into Transformation Competent S. thermophilus

Point mutations have been introduced in a pre-selected gene or location in the S. thermophilus genome according to the following protocol.

A mutated PCR fragment homologous to the selected region is made. This is made by two-step PCR in such a way that the desired point mutation(s) is placed approximately in the middle of an approximately 2 kb DNA fragment. This DNA fragment is identical to the target region, apart from the presence of the point mutation. This mutated homologous 2 kb fragment is then cloned into a vector, for instance the pCR 2.1-TOPO vector. The vector containing the 2 kb insert is linearised, e.g. using a restriction enzyme that has a unique cleavage site in the region opposite the insert. The resulting linear fragment is in general designed so as to have at least 1 kb of vector sequence flanking the 2 kb fragment at each end. The purpose of the non-homologous vector sequences is to protect the 2 kb fragment from degradation by nucleases present in the competent cell.

The linear DNA fragment described above (about 1 μg DNA fragment per ml of competent cells) is added to competent cells as above. Before spreading on agar plates the long chains of S. thermophilus cells are disrupted by using a Ultra-Turrax T25 mechanical blender (see Monnet et al 2004 J Dairy Sci 87, 1634). About 0.1-1% of the colonies growing on the plate will contain the desired point mutations. The same procedure can be used to make deletions or insertions of new genetic material (e.g. new genes and/or promoters).

When the above protocol was carried out, due to the high ratio of mutants to wild type cells (about 0.1-1% of the colonies growing on the plate contain the desired point mutations), we were able to identify bacteria containing the desired mutants by performing colony lift followed by hybridization with an oligonucleotide probe (20-30 nucleotides) homologous to the mutated region. This technique, together with the technique used to induce the competent state in S. thermophilus, make it possible to manipulate the genome of S. thermophilus with surgical precision. In principle no foreign DNA or selection markers needs to be introduced unless this is desired.

### RESULTS DISCUSSION

By using the highly efficient transformation procedure described above it is possible to introduce mutations into the genome of S. thermophilus without the use of a selectable antibiotic resistance marker. DNA fragments containing the desired insertion/deletion or point mutation(s) can be made by PCR or other molecular methods and cloned into a suitable plasmid. Following uptake of this construct by competent S. thermophilus LMG 18311 cells, targeted integration of the mutated region into the bacterial genome is mediated by ˜1000 bp flanking regions through double-crossover homologous recombination. Due to the high transformation efficiency transformants containing the sought after genotype against the background of wild type streptococci may be readily identified. Standard colony hybridization with a labelled oligonucleotide probe designed to specifically recognize mutants could be used for this purpose.

Before plating a mechanical blender (e.g. Ultra-Turrax model T25; Ika Labotechnik, Staufen, Germany) must be used to disrupt the long chains of S. thermophilus cells as described previously (Monnet et al., 2004, J. Dairy Sci., 87, p 1634-1640). After identification of the desired mutant it is easily cured of the unstable pXL helper plasmid by growth in the absence of erythromycin. S. thermophilus mutants made with this technique fulfil the safety criteria elaborated by Johansen (Johansen, 1999, in: “Encyclopedia of Food Microbiology”, Eds: Robinson et al., London, Academic Press, p 917-921), and should therefore attain “generally recognized as safe” (GRAS) status provided that DNA from non-GRAS organisms is not introduced into the genetically engineered strain.

In the present work we have shown that overexpression of ComX induces the competent state in S. thermophilus LMG 18311. An important question that remains to be answered, however, is how natural transformation is turned on spontaneously in this strain. Although it is possible that the mechanism controlling competence development has degenerated during adaptation to the dairy niche, it is more likely that spontaneous competence development in S. thermophilus LMG 18311 requires special, as yet undiscovered, growth conditions. The regulatory pathway controlling expression of the comX gene is so far unknown, but our results show that the gene is actively transcribed during early logarithmic phase when the XP strain is grown in THG medium at 37° C. In spite of this, transcription of the late genes under these conditions stayed very low, strongly indicating that ComX was prevented from accumulating to levels required for late gene expression by a regulatory mechanism operating at the posttranscription al level. It has been reported that the ClpP protease negatively controls ComX in S. pneumoniae (Chastanet et al., 2001, supra; Sung & Morrison, 2005, supra) and Streptococcus pyogenes (Opdyke et al., 2003, J. Bacteriol., 185, p 4291-4297), and it is therefore likely the same control mechanism is operating in S. thermophilus.

The fact that overexpression of ComX efficiently induces expression of the late genes, suggests that the system that negatively controls the accumulation of ComX become saturated under these circumstances. In sum, the data indicate that spontaneous competence induction in S. thermophilus requires the joint action of at least two converging regulatory pathways. However, in contrast to other streptococci that have been shown to be competent for natural transformation, a quorum-sensing system of the ComCDE type does not seem to be involved.

Considering the high degree of degeneracy detected in the genome of S. thermophilus it is remarkable that the genes involved in natural transformation have remained intact. Bolotin et al. (Bolotin et al., 2004, Nat. Biotechnol., 22, p 1554-1558) found that 10% of the genes in S. thermophilus strains LMG 18311 and CNRZ 1066 are non-functional pseudogenes, and concluded that these strains have adapted to the dairy niche mainly through loss-of-function events. The intactness of the late competence genes in strain LMG 18311 strongly indicates that even in a constant milk environment natural competence provides a selective advantage. Indeed, evidence of lateral gene transfer from other dairy bacteria to S. thermophilus LMG 18311 has been reported. A 17-kb mosaic region found within the pepD gene contains fragments with high homology to corresponding sequences in Lactobacillus bulgaricus and Lactococcus lactis, species that will come into close contact with S. thermophilus during fermentation of yoghurt and cheeses, respectively (Bolotin et al., 2004, supra). The fact that S. thermophilus now has been found to be naturally transformable opens up the possibility that at least some of the observed gene transfer events have taken place by this mechanism.

Similar to S. thermophilus, the important pathogens, S. pyogenes and Streptococcus agalactiae, have traditionally been considered non-competent even though they possess the ComX regulon. It is known that members of these species are involved in frequent recombinational exchanges and harbour genes with mosaic structures, features that have been attributed to lateral gene transfer mediated by conjugation or transduction (Feil et al., 2001, Proc. Natl. Acad. Sci. USA, 98, p 182-187; Kapur et al., 1995, Mol. Microbiol., 16, p 509-519; Brochet et al., 2006, Microbes Infect., E-pub). In light of the results presented here, however, an active role of natural genetic transformation in shaping the genomes of S. pyogenes and S. agalactiae cannot be excluded.

1. A method of producing transformation competent bacteria, comprising the steps of:
(i) transforming a bacteria that is not naturally transformation competent with a plasmid, wherein said plasmid comprises a comX gene sequence encoding a ComX protein or functional part or derivative or variant thereof under the regulatory control of a promoter which is inducible by a transcription initiator,
(ii) contacting said transformed bacteria with said transcription initiator to initiate transcription of said comX gene sequence.

2. The method of claim 1 wherein said bacteria is S thermophilus.

3. The method of claim 1, wherein said bacteria is S thermophilus strain LMG 18311.

4. The method of claim 1, wherein said comX gene sequence is from Streptococcus.

5. The method of claim 4 wherein said Streptococcus is S. thermophilus.

6. The method of claim 1, wherein said ComX gene sequence comprises:
(i) the nucleotide sequence of SEQ ID NO:1; or
(ii) a portion thereof; or
(iii) a sequence which hybridizes to said sequence or portion thereof under non-stringent binding conditions and washing under conditions of high stringency; or
(iv) a sequence which exhibits at least 80% sequence identity to said sequence or portion thereof; or
(v) a sequence complementary to any of the aforesaid sequences.

7. The method of claim 1, wherein said ComX gene sequence encodes an amino acid sequence which comprises:
(i) the amino acid sequence of SEQ ID NO:2; or
(ii) a portion thereof; or
(iii) a sequence which exhibits at least 80% sequence identity to said sequence or portion thereof.

8. The method of claim 6 or 7 wherein the portion of said ComX gene sequence comprises at least 400 bases of said sequences.

9. The method of claim 1, wherein said promoter that is induced by the transcription initiator is a bacteriocin promoter.

10. The method of claim 9 wherein said bacteriocin promoter is the bacteriocin promoter of S thermophilus.

11. The method of claim 1 wherein said promoter comprises:
(i) the nucleotide sequence of SEQ ID NO:3; or
(ii) a portion thereof; or
(iii) a sequence which hybridizes to said sequence or portion thereof under non-stringent binding conditions and washing under conditions of high stringency; or
(iv) a sequence which exhibits at least 80% sequence identity to said sequence or portion thereof; or
(v) a sequence complementary to any of the aforesaid sequences.

12. The method of claim 1 wherein said transcription initiator comprises:
(i) the amino acid sequence of SEQ ID NO:4; or
(ii) a portion thereof; or
(iii) a sequence which exhibits at least 80% sequence identity to said sequence or portion thereof.

13. The method of claim 1, wherein said transcription initiator comprises:
(i) the amino acid sequence SEQ ID NO:5; or
(ii) a portion thereof; or
(iii) a sequence which exhibits at least 80% sequence identity to said sequence or portion thereof.

14. The method of claim 1, wherein said plasmid further comprises a reporter gene under the control of a promoter.

15. The method of claim 14, wherein the reporter gene is under the control of a promoter which is induced as a result of expression of the comX gene.

16. The method of claim 15 wherein said promoter is the late gene comEC promoter.

17. A method of producing transformation competent S. thermophilus bacteria, comprising at least the steps of:
(i) transforming a S. thermophilus bacteria with a plasmid, wherein said plasmid comprises a comX gene sequence which comprises:
(a) the nucleotide sequence of SEQ ID NO:1; or
(b) a portion thereof; or
(c) a sequence which hybridizes to said sequence or portion thereof under non-stringent binding conditions and washing under conditions of high stringency; or
(d) a sequence which exhibits at least 80% sequence identity to said sequence or portion thereof; or
(e) a sequence complementary to any of the aforesaid sequences;
under the regulatory control of a promoter comprising:
(a) the nucleotide sequence of SEQ ID NO:3; or
(b) a portion thereof; or
(c) a sequence which hybridizes to said sequence or portion thereof under non-stringent binding conditions and washing under conditions of high stringency; or
(d) a sequence which exhibits at least 80% sequence identity to said sequence or portion thereof; or
(e) a sequence complementary to any of the aforesaid sequences;
which is inducible by a transcription initiator comprising:
(a) the amino acid sequence of SEQ ID NO:4; or
(b) a portion thereof; or
(c) a sequence which exhibits at least 80% sequence identity to said sequence or portion thereof,
optionally further comprising a reporter gene under the control of said promoter; and
(ii) contacting said transformed bacteria with said transcription initiator to initiate transcription of said comX gene sequence.

18. The method of claim 1, wherein said plasmid is unstable in the bacteria.

19. The method of claim 1, wherein said plasmid is selected from
(i) the pXL plasmid,
(ii) a plasmid having at least 80% sequence identity to the pXL plasmid, and
(iii) a plasmid comprising a promoter and comX sequence with at least 80% identity to the promoter and comX sequence of the pXL plasmid.

20. A plasmid comprising a comX gene sequence encoding a ComX protein or functional part or derivative or variant thereof under the regulatory control of a promoter which is inducible by a transcription initiator.

21. The plasmid of claim 20, wherein said plasmid further comprises a reporter gene under the control of a promoter.

22. The plasmid of claim 20, wherein said comX gene sequence comprises:
(i) the nucleotide sequence of SEQ ID NO:1, or a portion thereof; or a sequence which hybridizes to said sequence or portion thereof under non-stringent binding conditions and washing under conditions of high stringency, or a sequence which exhibits at least 80% sequence identity to said sequence or portion thereof; or a sequence complementary to any of the aforesaid sequences; or
(ii) the amino acid sequence of SEQ ID NO:2; or a portion thereof; or a sequence which exhibits at least 80% sequence identity to said sequence or portion thereof.

23. The plasmid of claim 20 wherein said promoter is a bacteriocin promoter.

24. The plasmid of claim 20 which is selected from
(i) the pXL plasmid,
(ii) a plasmid having at least 80% sequence identity to the pXL plasmid, and
(iii) a plasmid comprising a promoter and comX sequence with at least 80% identity to the promoter and comX sequence of the pXL plasmid.

25. Bacteria transformed with the plasmid of claim 20, wherein said bacteria is not naturally transformation competent prior to transformation.

26. The bacteria of claim 25 which is S. thermophilus.

27. A method of producing transformation competent bacteria, comprising contacting the transformed bacteria of claim 25 with a transcription initiator to initiate transcription of said comX gene sequence.

28. Transformation competent bacteria produced according to the method of claim 1.

29. A method of producing a mutant bacteria comprising the steps of:
(i) contacting the transformation competent bacteria of claim 28 with homologous DNA comprising a mutation under conditions to allow transformation of said bacteria with said homologous DNA.

30. The method of claim 29 further comprising the steps of selecting and/or amplifying the mutant bacteria thus generated.

31. The method of claim 29, wherein the plasmid is unstable in the bacteria and said method further comprises the step of culturing said bacteria under conditions that cause said unstable plasmid to be lost from said bacteria.

32. The method of claim 1, wherein at least one step of said method is performed in growth medium that comprises one or more of heart infusion, neopeptone (or peptonen or peptone e.g. casein or yeast peptone), dextrose, sodium chloride, disodium phosphate, glucose and sodium carbonate.

33. The method of claim 32 wherein said growth medium comprises about 0.5 to about 10 g per litre heart infusion, about 10 to about 50 g per litre neopeptone, peptonen or peptone, about 0.5 to about 10 g per litre dextrose, about 0.5 to about 10 g per litre sodium chloride, about 0.1 to about 2 g per litre disodium phosphate, about 1.0 to about 5.0 g per litre sodium carbonate and about 0.1 to about 10% glucose.

34. Mutant bacteria produced according to the method of claim 29.

35. A method of producing a food product comprising at least the step of fermentation using the mutant of claim 34.

36. Food products generated by the method of claim 35.

37. The method of claim 14 further comprises the step of selecting and/or amplifying the transformation competent bacteria.

38. The method of claim 17, wherein said plasmid is unstable in the bacteria.

39. The method of claim 17, wherein said plasmid is selected from
(i) the pXL plasmid,
(ii) a plasmid having at least 80% sequence identity to the pXL plasmid, and
(iii) a plasmid comprising a promoter and comX sequence with at least 80% identity to the promoter and comX sequence of the pXL plasmid.

40. Transformation competent bacteria produced according to the method of claim 17.

41. The method of claim 17, wherein at least one step of said method is performed in growth medium that comprises one or more of heart infusion, neopeptone (or peptonen or peptone e.g. casein or yeast peptone), dextrose, sodium chloride, disodium phosphate, glucose and sodium carbonate.

42. The method of claim 17 further comprises the step of selecting and/or amplifying the transformation competent S. thermophilus bacteria.
